(19) United States
(12) Patent Application Publication
(10) Pub. No.: US 2023/0105338 A1
(43) Pub. Date: Apr. 6, 2023

(54) CONTACT-FREE BIOSENSOR

(71) Applicant: GLOBALFOUNDRIES U.S. Inc., Malta, NY (US)

(72) Inventors: Bartlomiej J. Pawlak, Leuven (BE); Aaron L. Vallett, Jericho, VT (US); Siva P. Adusumilli, South Burlington, VT (US); Mark D. Lagerquist, Colchester, VT (US)

(73) Assignee: GLOBALFOUNDRIES U.S. Inc., Malta, NY (US)

(21) Appl. No.: 17/493,969

(22) Filed: Oct. 5, 2021

(51) Int. Cl. G01N 33/487; G01N 27/414

(52) U.S. Cl. CPC G01N 33/4875; G01N 33/48707; G01N 27/4145

(57) ABSTRACT

A structure includes a first layer having a recess. The structure further includes an intermediate layer contacting the first layer and a contact-free biosensor aligned above the recess. The portion of the intermediate layer that is positioned along the recess separates the contact-free biosensor from the recess.

### BACKGROUND

### Field of the Invention

The present disclosure relates to sensors and, more specifically, to biosensors.

### Description of Related Art

Various types of biosensors exist, and one type of particular interest are radio-frequency (RF), microwave (MW), millimeter wave (mmW) and terahertz (THz) biosensors (all of which are referred to herein as biosensors for simplicity of discussion). Such biosensors treat biological tissue as a dielectric substance having a unique dielectric signature, which can be characterized by frequency dependent parameters such as permittivity and conductivity, and can operate on the principle of resonator-based biosensors. With biosensors, capacitive sensing is sometimes employed where changes in permittivity are reflected as changes in capacitance, through components like interdigitated electrodes, resonators and microstrip structures.

Such biosensors can subject biomatter to electro-mechanical (EM) waves and this allows biosensors to observe the unique permittivity signatures of the biomatter without having to directly contact the fluid under test. Permittivity (in the framework of electromagnetics) is a fundamental material property that describes how a material will affect, and be affected by, a time-varying electromagnetic field.

### SUMMARY

According to one embodiment herein, a structure includes a first layer, where the first layer includes a recess. The structure further includes an intermediate layer contacting the first layer and a contact-free biosensor aligned above the recess. A portion of the intermediate layer is positioned along the recess, and the portion of the intermediate layer that is positioned along the recess separates the contact-free biosensor from the recess.

According to another embodiment herein, a structure includes a first layer, where the first layer includes a recess. The structure further includes an intermediate layer contacting the first layer and a plurality of contact-free biosensors aligned above the recess. A portion of the intermediate layer is positioned along the recess and the portion of the intermediate layer that is positioned along the recess separates the contact-free biosensors from the recess.

According to a further embodiment herein a method forms a first layer to include a recess, forms an intermediate layer to contact the first layer, and forms a contact-free biosensor aligned above the recess. A portion of the intermediate layer is formed to be positioned along the recess, and the portion of the intermediate layer that is positioned along the recess is formed to separate the contact-free biosensor from the recess.

### BRIEF DESCRIPTION OF THE DRAWINGS

The embodiments herein will be better understood from the following detailed description with reference to the drawings, which are not necessarily drawn to scale and in which:

FIG. 1 is a cross-sectional schematic diagram illustrating an RF device according to embodiments herein;

FIG. 2 is a top view schematic diagram illustrating an RF device according to embodiments herein;

FIGS. 3-6 are cross-sectional schematic diagrams illustrating contact-free biosensors according to embodiments herein;

FIGS. 7-8 are top view schematic diagram illustrating contact-free biosensors according to embodiments herein;

FIGS. 9-10 are cross-sectional schematic diagrams illustrating contact-free biosensors according to embodiments herein;

FIGS. 11-12 are top view schematic diagram illustrating contact-free biosensors according to embodiments herein; and

FIGS. 13-27 are cross-sectional schematic diagrams illustrating processes that form contact-free biosensors according to embodiments herein.

### DETAILED DESCRIPTION

Bio-sensing of liquids with clear identification is a major issue for lab-on-a-chip solutions. As noted above, with biosensors, capacitive sensing is sometimes employed where changes in permittivity are reflected as changes in capacitance, through components like interdigitated electrodes, resonators and microstrip structures. For example, with some biosensors the radio-frequency (RF) spectrum of the droplet deposited on the transmission line is detected. Others can use optical detection and electrical detection.

Contact-free biosensors herein can be radio-frequency (RF), microwave (MW), millimeter wave (mmW) and terahertz (THz) microfluidic biosensors and can subject biomatter to electro-mechanical (EM) waves and this allows such contact-free biosensors to observe the unique permittivity signatures of the biomatter. Permittivity (in the framework of electromagnetics) is a fundamental material property that describes how a material will affect, and be affected by, a time-varying electromagnetic field.

The devices and methods herein perform time-varying electromagnetic field sensing to obtain a permittivity signature of molecular species by supplying the bio-liquid via microfluidic channels created in a silicon substrate. Specifically, with the devices and methods herein, transistors or passive devices (e.g., antenna, transmission line, etc.) are located in close vicinity of a microfluidic channel to obtain the signature of molecular species without direct contact between the sensor and the molecular species (contact-free sensing).

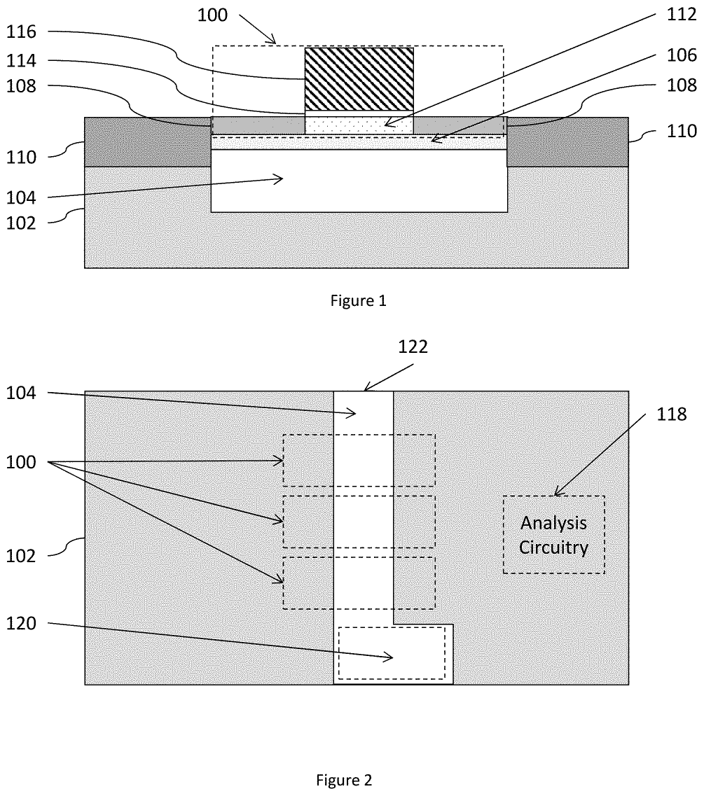

FIG. 1 is a cross-sectional view that illustrates aspects of some exemplary structure herein, which is part of an overall lab-on-a-chip device, that includes (among other components) a microfluidic channel (which can be a trench, groove, cutout, opening, open space, etc., and that is referred to herein generically as a recess 104 for simplicity of discussion) adjacent a transistor. In FIG. 1, the recess is located in a first layer 102. The first layer 102 can be a bulk wafer substrate or any other type of layer of sufficient structural rigidity, for example.

A field effect transistor 100 is shown in FIG. 1 as a representative transistor or “active” contact-free biosensor; however, any type of transistor or active device could be utilized, and the field effect transistor 100 shown in FIG. 1 is intended to be representative of all forms of transistors and active devices that can perform non-contact time-varying electromagnetic field sensing. In FIG. 1, the exemplary transistor 100 (generally enclosed within a broken line box in FIG. 1) includes a channel region 112 bordered by source/drain regions 108. A gate oxide 114 separates the channel region from a gate conductor 116. Shallow trench isolation regions 110 electrically insulate the transistor 100 from adjacent devices.

As shown in FIG. 1, an intermediate layer 106 contacts the first layer 102. The intermediate layer 106 can be, for example, a buried oxide (BOX) layer, a silicon layer, a silicon oxide layer, and/or a silicon germanium layer, etc. In the example in FIG. 1, the intermediate layer 106 is a BOX layer. The transistor 100 is positioned on the intermediate layer 106 aligned above the recess 104. Specifically, the transistor 100 is positioned relative to the recess 104 in a location to detect characteristics of a fluid under test within the recess 104 using a time-varying electromagnetic field generated and/or received by the radio frequency devices. Further, the thickness of the intermediate layer 106 has a size that allows the time-varying electromagnetic fields to pass between the transistor 100 and the recess 104, and the size of the thickness of the intermediate layer 106 does not restrict the time-varying electromagnetic fields from passing between the transistor 100 and the recess 104. Therefore, the portion of the intermediate layer 106 that is positioned along the recess 104 separates the transistor 100 from the recess 104 but does not substantially prevent time-varying electromagnetic fields from passing.

All elements of the transistor 100 are formed using conventional processing, whether currently known or developed in the future. Additionally, contacts and additional wiring layers are generally formed above the transistor 100 in a multi-layer laminated structure to provide electrical contacts to the various components of the transistor 100.

FIG. 2 is a top (plan) view of the first layer 102 that shows the recess 104 therein. Broken line boxes are used to represent locations of various transistors 100 that can be positioned in layers of the lab-on-a-chip device above the first layer 102 and aligned above (aligned with) the recess 104. For purposes herein, one item is “aligned above” or “aligned with” another item with respect to the arbitrarily named “top” and “bottom” of the lab-on-a-chip device (e.g., the arbitrary orientation shown in FIG. 1), and such top-down view alignment is shown for example using the top view in FIG. 2. As shown in FIG. 1, the recess 104 has sidewalls and a bottom and an open top, and the transistor 100 is “aligned above” the bottom of the recess 104 or, stated differently, the transistor 100 is positioned with respect to the recess 104 such that a line extending perpendicularly from the center of the bottom of the recess 104 intersects the transistor 100. Similar “alignment” language is used herein to identify the same positional alignment relationship between other features also.

Broken line box 120 in FIG. 2 identifies an area of the recess 104 that is a fluid receptacle, which is an enlarged region sized to permit droplets of a fluid under test to be supplied to the recess 104. Item 122 shows that the end of the recess 104 is a fluid outlet allowing fluid to flow by capillary action, electrical/magnetic attraction, gravity, or by use of mechanical devices, from the fluid receptacle 120, along the recess 104, to the fluid outlet 122. The fluid receptacle 120 and fluid outlet 122 of the recess 104 can be in any locations that permit the fluid under test to be supplied from an exterior of the device and to flow through the recess 104 past the transistor 100, and such are only shown in exemplary positions in FIG. 2 but such components can be in any appropriate locations.

A broken line box is also used to represent a location of conventional analysis circuitry 118 used with conventional lab-on-a-chip devices that can be formed in layers over the first layer 102. The analysis circuitry 118 is connected to the transistors 100 (in upper layers of the lab-on-a-chip device that are not shown in FIG. 2) and the analysis circuitry 118 automatically identifies characteristics of the liquid within the recess 104 using the transistor(s) 100. Further, the analysis circuitry 118 reports the results of the analysis using one or more of many different types of communication and identification interfaces (visible, wired, wireless, etc.).

The analysis circuitry 118 in combination with the transistor(s) 100 allows the lab-on-a-chip device to fully and automatically analyze, and provide reports of, the fluid within the recess 104. In operation, a fluid under test (e.g., biological fluid) is supplied to the fluid receptacle 120 of the recess 104. The fluid under test travels to fill the recess 104 (through capillary forces (capillary action), microscopic pumps (microelectronic mechanical structures (MEMS)). The one or more transistors 100 evaluate the fluid under test by transmitting and receiving a time-varying electromagnetic field to evaluate the permittivity signatures of the fluid under task.

The use of multiple transistors 100 as shown in FIG. 2 provides redundancy to accommodate defective transistors, provides like comparisons to automatically validate results, allows averaging of results, etc. For example, the transistors 100 shown in FIG. 2 can all be identical and can all perform the same analysis on the same fluid under test. If one of the transistors 100 fails, the remaining transistors can take the failed transistor's place. If at least two of the transistors 100 operate properly, the results of one can be compared to the other(s) to verify that the results are correct. Additionally, results from different transistors 100 can be averaged (or other statistical measures can be observed) to provide a higher confidence analysis.

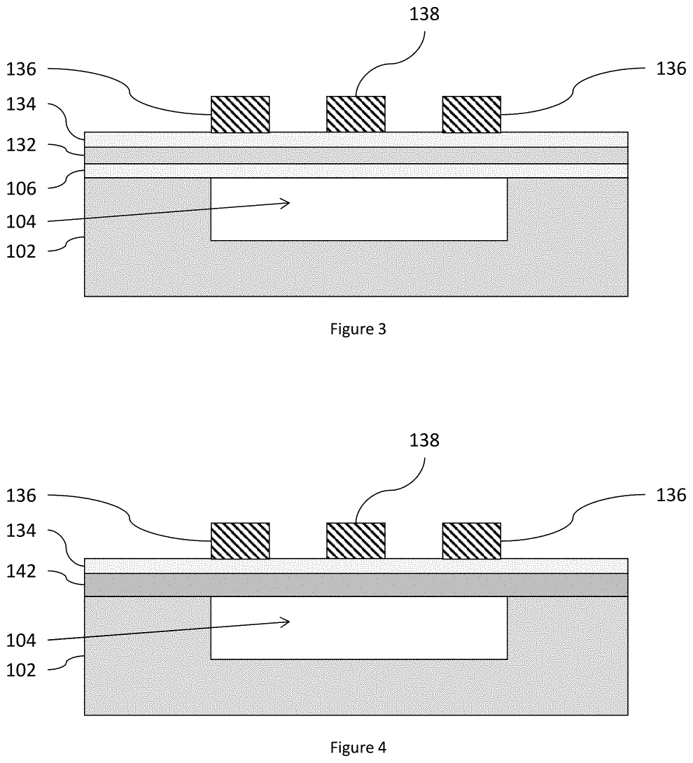

In some examples herein, the contact-free biosensor devices herein can be any form of passive device, such as transmission line(s), antenna(s), etc. Therefore, as shown in FIGS. 3-4, a conductor (a transmission line or antenna 138) can be positioned aligned above the recess 104. Items 136 in FIGS. 3-4 are optional ground lines that assist/focus the electromagnetic field generated and/or received by the transmission line or antenna 138.

As shown in FIG. 3, multiple intermediate layers can be used so that the intermediate layer can also include (in addition to layer 106) additional layers 132, 134 which can be, for example, silicon 132 and silicon dioxide 134. In FIG. 4, the intermediate layer is also multiple layers and includes (in addition to layer 106), for example, a silicon germanium layer 142 and the silicon dioxide layer 134. The layer or layers that are used as the intermediate layer are selected to operate with the specific active or passive device being used; however, in all structures the active or passive device (e.g., 100, 138, etc.) is positioned relative to the recess 104 in a location to detect characteristics of a fluid under test within the recess 104 using a time-varying electromagnetic field generated and/or received by the contact-free biosensor devices herein. In other words, the multi-layer intermediate layer (e.g., 106, 132, and 134; or 142 and 134) has a size that allows the time-varying electromagnetic fields to pass between the active or passive devices 100, 138 and the recess 104, and the size of the thickness of the intermediate layer 106 does not restrict the time-varying electromagnetic fields from passing between the active or passive devices 100, 138 and the recess 104.

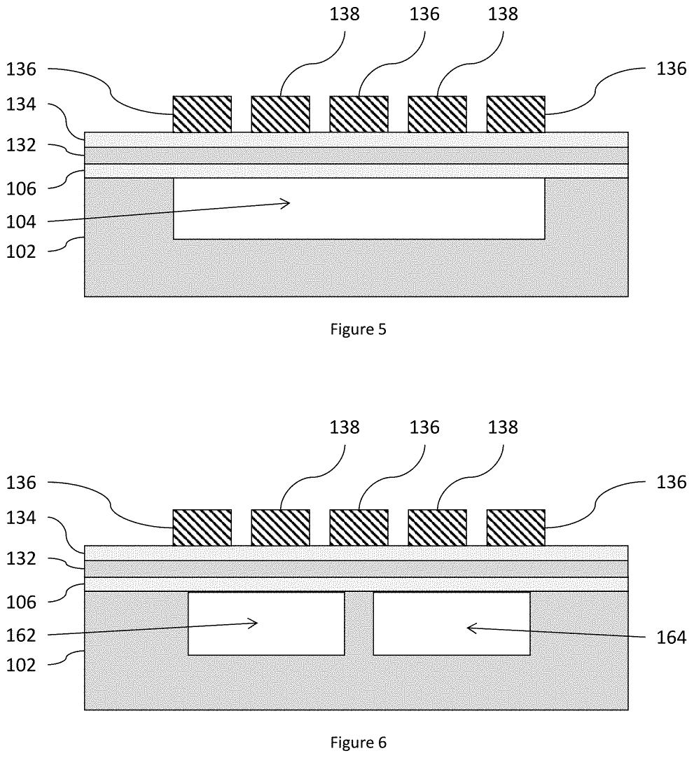

FIG. 5 shows that multiple transmission lines or antennas 138 can be positioned adjacent a single recess 104 and that additional ground lines 136 can be used to assist/focus the electromagnetic field generated and/or received by the transmission lines or antennas 138. Further, multiple transmission lines or antennas 138 can provide broader frequencies which can allow the lab-on-a-chip device to perform more granular analysis and wider field analysis.

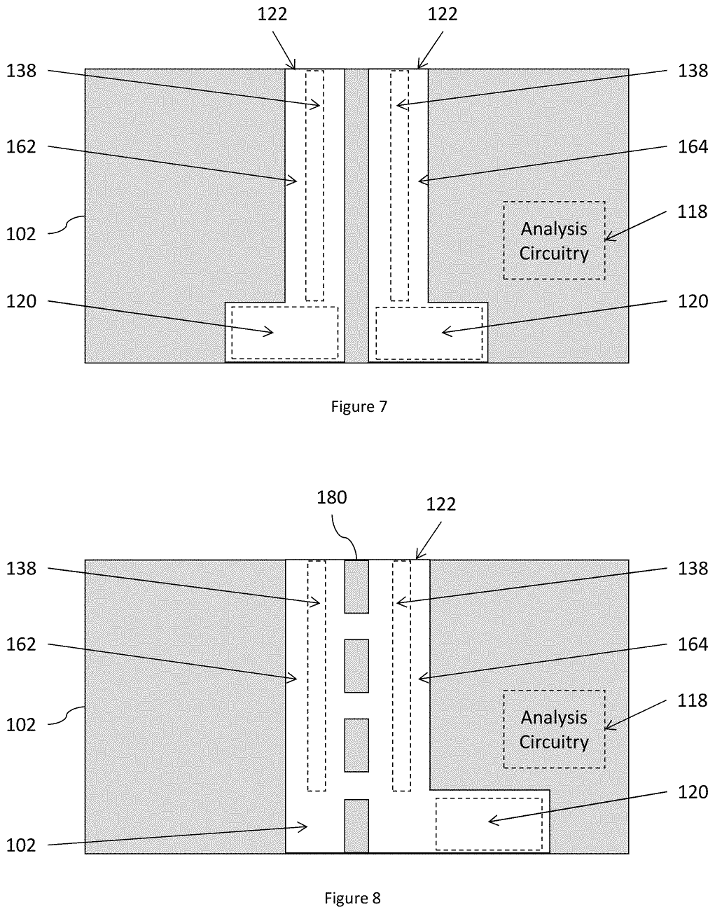

FIG. 6 shows that multiple recesses 162, 164 can be used and the same is shown in top view in FIG. 7 where the positions of the transmission lines or antennas 138 are shown adjacent and aligned above the recesses 162, 164. In the example shown in FIGS. 6-7, a single transmission line or antenna 138 can be aligned above each recess 162, 164.

The use of multiple transmission lines or antennas 138 and/or multiple recesses 162, 164, as shown in FIGS. 5-7, similarly provides redundancy to accommodate defective transmission line or antenna 138, provides like comparisons to automatically validate results, allows averaging of results, etc., as discussed above. Further the use of a separate recess 162, 164 allows a control fluid to be supplied to one recess while a fluid under test is supplied to the other recess. This allows the structures herein to simultaneously analyze the fluid under test and the control fluid to provide a reference result from the control fluid that can be compared to the analysis result of the fluid under test.

As shown in FIG. 8, a plurality of dividers 180 can be positioned within the recess 104. The dividers 180 can be used to create turbulence within the fluid under test and or can be used to control capillary action. These controls allow the various analyses that are performed to be fine-tuned and customized for the specific type, viscosity, temperature, etc., of liquid that makes up the fluid under test.

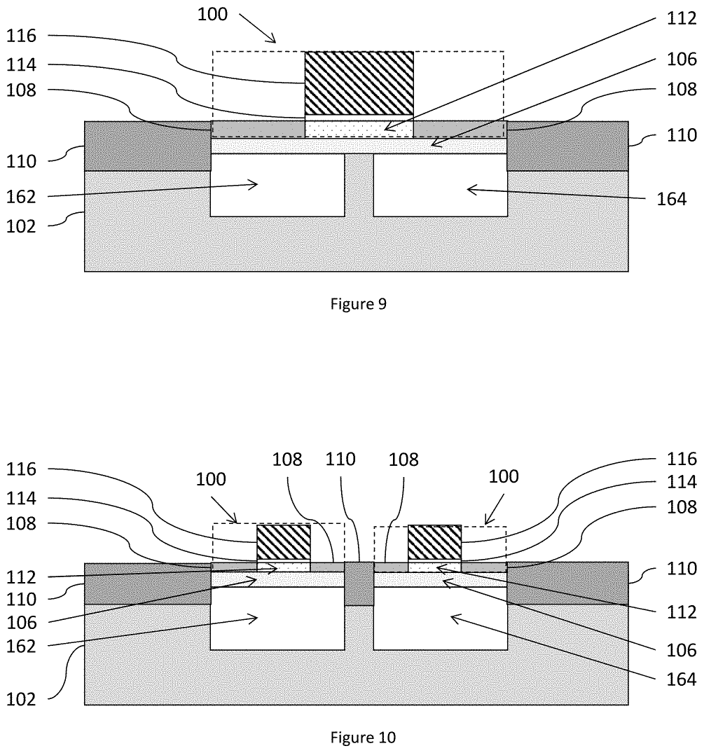

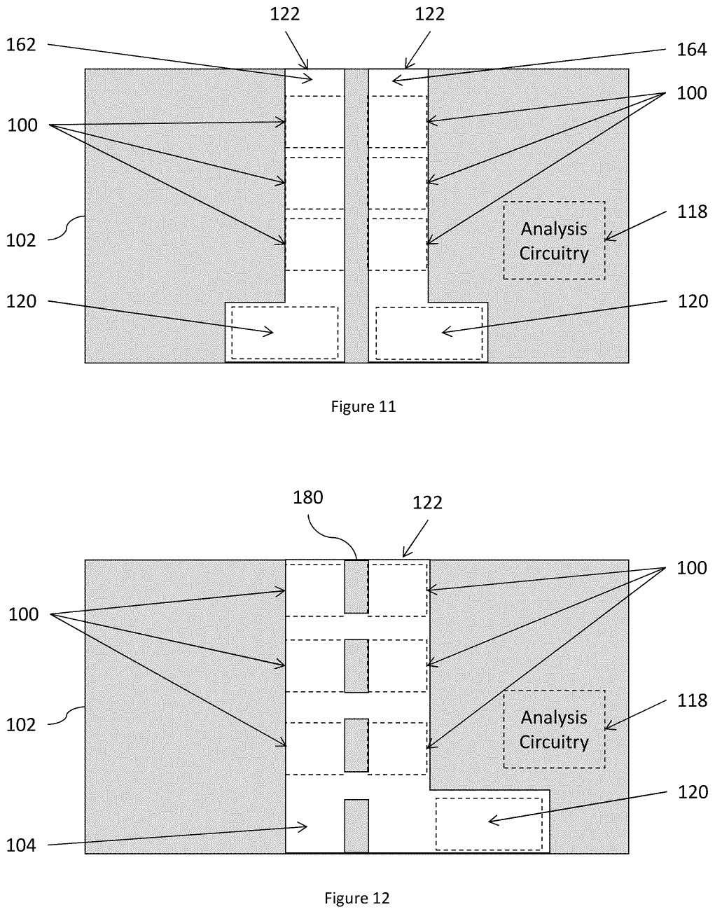

FIG. 9 shows that a single transistor 100 can be aligned above more than one recess 162, 164 and FIG. 10 shows that multiple transistors 100 can be aligned above multiple recesses 162, 164, potentially with each transistor 100 being aligned with a single corresponding recess. FIGS. 11-12 similarly show top views of the first layer 102 and the positions of the multiple transistors 100 aligned above multiple recesses 162, 164 (FIG. 11) and aligned above a single recess 102 with dividers 180 (FIG. 12). Again, the use of multiple transistors and/or multiple recesses provides redundancy, like comparisons to automatically validate results, and averaging of results, etc., as discussed above.

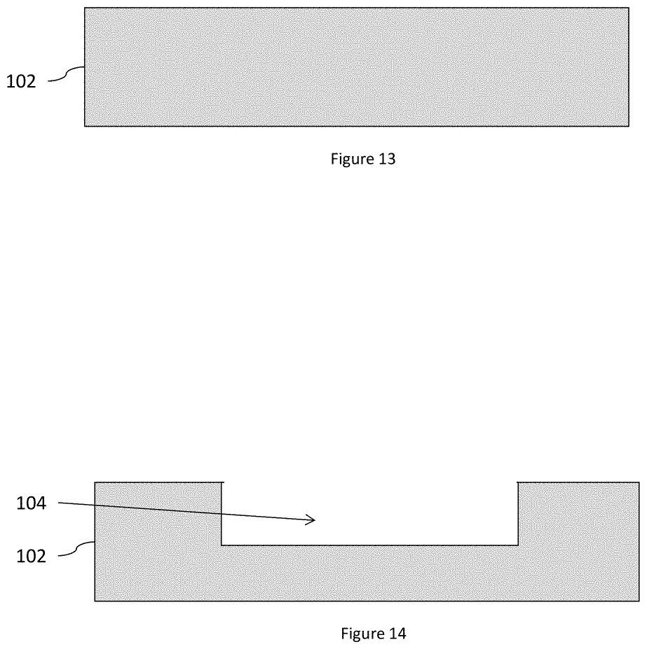

FIGS. 13-21 illustrate exemplary methods herein that form a first layer 102 to include at least one recess 104 and to form an intermediate layer 106 to contact the first layer 102. While many different processes can be used to form the recess, the one shown in FIGS. 13-21 is a representative example of front-end-of-line (FEOL) processes, whether currently known or developed in the future.

More specifically, in FIG. 13 a substrate 102 is supplied. In FIG. 14, the recess 104 is patterned in the substrate 102 using any conventional patterning technique (e.g., masking and etching, laser ablation, etc.). Note that while the recess 104 is shown as being rectangular and having straight sidewalls, a bottom and an open top; in other embodiments the recess 104 could be rounded, have a U-shape, C-shape, V-shape, bottle shape, etc., and all such cross-sectional shapes are intended to be illustrated by the recess 104. Further, the recess 104 is sized and shaped to be a microfluidic channel. Thus, in such processing the recess 104 is sized to nanometer specifications, is shaped to avoid sharp bends, and is formed to dimensions that will promote capillary or gravitational action given the specific characteristics of the fluid under test (e.g., viscosity, electrical charge, surface tension, etc.).

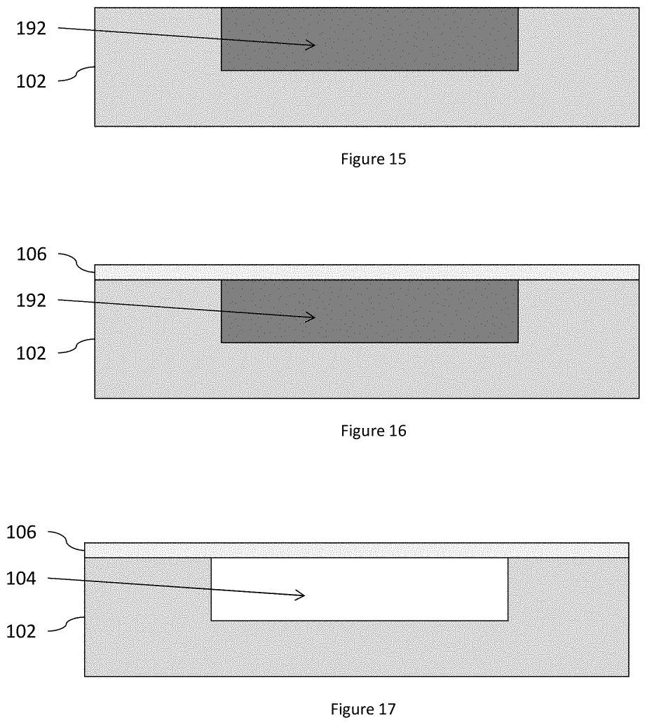

In FIG. 15 the recess 104 is filled (using deposition or growth processes) with a sacrificial material 192, and the sacrificial material can be planarized after formation if needed. The sacrificial material 192 can be selectively dissolved with respect to the materials of the remaining structure to allow the sacrificial material to be removed without affecting the remaining structure.

In FIG. 16, at least one intermediate layer 106 is formed (e.g., grown, deposited, etc.). Here the presence of the sacrificial material 192 prevents the intermediate layer 106 from entering or being formed in the recess 104. In FIG. 17, a selective material removal process is performed to selectively remove the sacrificial material 192 without affecting the remaining structure. With the recess 104 and intermediate layer 106 now in place, one or more active or passive devices 100, 138 can be formed aligned above the recess 104.

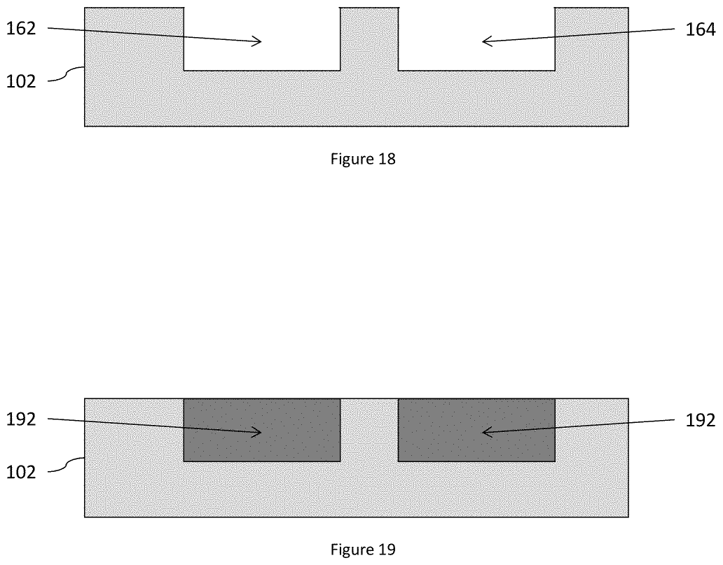

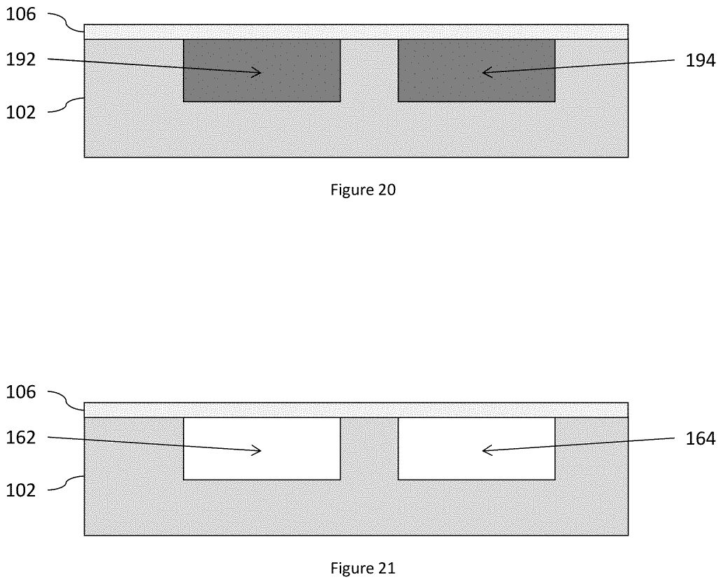

FIGS. 18-21 show similar processing for multiple recesses 162, 164. Thus, in FIG. 18, recesses 162, 164 are patterned in the substrate supplied in FIG. 13 again using any conventional patterning technique (e.g., masking and etching, etc.). In FIG. 19 the recesses 162, 164 are filled with the sacrificial material 192, which can be planarized later if needed. In FIG. 20, at least one intermediate layer 106 is formed (e.g., grown, deposited, etc.). Again, the presence of the sacrificial material 192 prevents the intermediate layer 106 from filling the recesses 162, 164. In FIG. 21, a selective material removal process is again performed to selectively remove the sacrificial material 192 without affecting the remaining structure. With the recesses 162, 164 and intermediate layer 106 now in place, one or more active 100 (or passive 138, represented by item 100 in FIG. 21) devices can be formed aligned above the recesses 162, 164, using conventional processing.

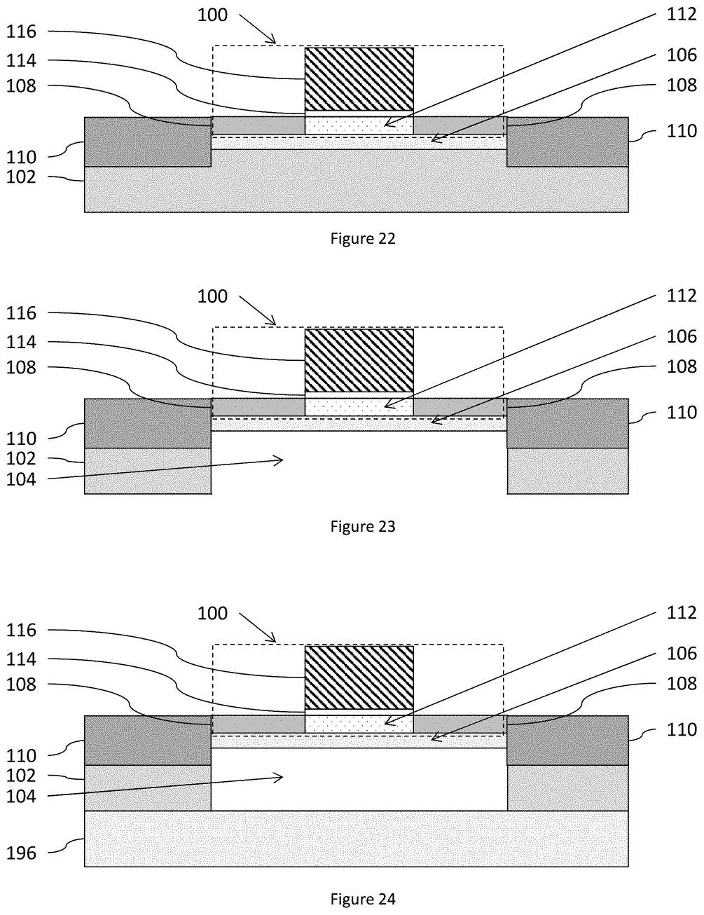

While FIGS. 13-21 show FEOL formation of the recesses 104, 162, 164, such can also be formed in back-end-of line (BEOL) processing, as shown in FIGS. 22-27. More specifically, FIG. 22 shows a completed transistor 100 formed using conventional processing on the substrate 102 that has not yet been patterned and, therefore, the recess 104 is not yet present. Any conventional processing can be used to from the transistor 100 and the transistor 100 is representative of all active or passive devices 100, 138 described herein.

In FIG. 23, the recess 104 is patterned in the bottom or backside of the substrate 102 using any conventional patterning technique (e.g., masking and etching, laser ablation, etc.). The bottom or backside of the substrate is the side opposite the location where the transistor is formed 100 (which is sometimes referred to as the top of the substrate 102, e.g., the top is opposite the bottom or backside of the substrate 102). The patterning process is stopped when the intermediate layer 106 is reached. Again, while the recess 104 is shown as being rectangular and having straight sidewalls, a bottom and an open top; in other embodiments the recess 104 could be rounded, have a U-shape, C-shape, V-shape, bottle shape, etc., and all such cross-sectional shapes are intended to be illustrated by the recess 104. Also, the recess 104 is sized and shaped to be a microfluidic channel. Thus, in such processing the recess 104 is sized to nanometer specifications, is shaped to avoid sharp bends, and is formed to dimensions that will promote capillary or gravitational action given the specific characteristics of the fluid under test (e.g., viscosity, electrical charge, surface tension, etc.).

As shown in FIG. 24, a second substrate 196 is attached to the backside of the substrate 102, using any conventional attachment processing including bonding (e.g., thermo-compression bonding), etc. The second substrate 196 forms the fourth side of the recess 104 and seals off the recess 104. The second substrate 196 can be the same as, or different from, the substrate 102 and is generally a bulk insulator with structural rigidity (e.g., wafer, etc.).

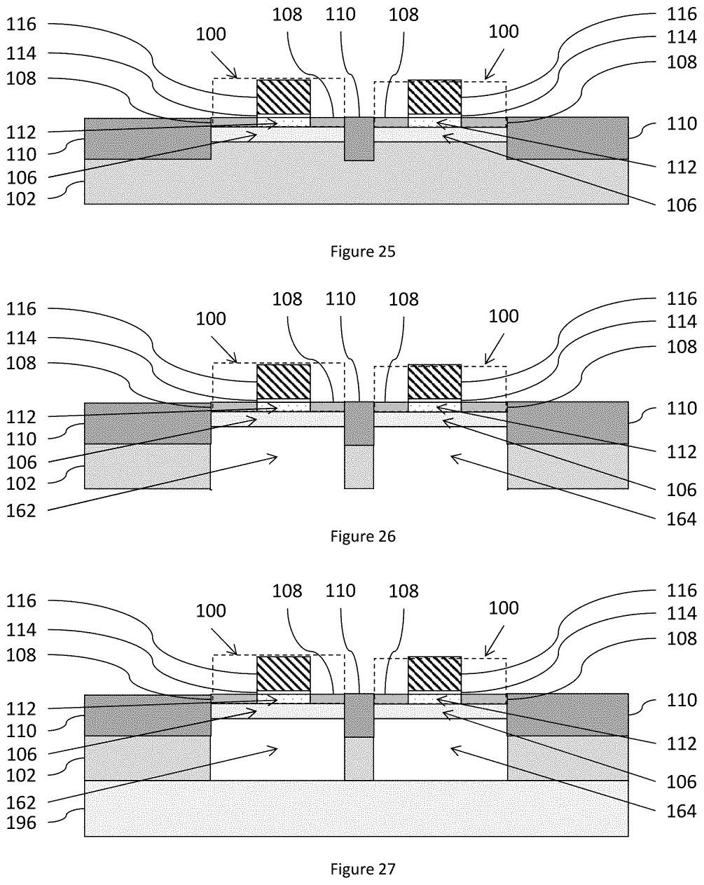

FIGS. 25-27 show similar processing for multiple recesses 162, 164. Thus, in FIG. 25, one or more active 100 (or passive 138, represented by item 100 in FIG. 25) devices are formed on the substrate 102 that has not been patterned. In FIG. 26, recesses 162, 164 are patterned in the substrate 102 again using any conventional patterning technique (e.g., masking and etching, etc.). In FIG. 27, a second substrate 196 is attached to the backside of the substrate 102, again using any conventional attachment processing including bonding, etc.

The above-described structures and methods provide a location (e.g., recess(es) 104, 162, 164) for a fluid under test to be positioned within close enough proximity of an active or passive contact-free biosensor 100, 138 to allow time-varying electromagnetic field sensing, while the intermediate layer (e.g., 106 alone; 106, 132, and 134; or 142 and 134, etc.) prevents the fluid under test from coming into direct contact with the active or passive device(s) 100, 138. Therefore, these structures and methods provide a contact-free biosensor that is simply and easily formed during FEOL or BEOL processing. Specifically, FEOL and BEOL processes are mature technologies and all existing sophisticated FEOL and BEOL techniques can be utilized to inexpensively and accurately achieve the needed shape, size, etc., components to accommodate the fluid under test that will be utilized with the lab-on-a-chip device.

Further, by forming the recess 104 in the bulk substrate 102 vertical space is saved compared to devices that locate microfluid channels on tops or in the middle of lab-on-a-chip devices. Forming the recess 104 in the substrate 102 also simplifies recess 104 location and associated processing because the bulk substrate 102 typically does not contain any components and the recess(es) can be formed in any desired location while, in contrast, channels formed in upper layers of lab-on-a-chip structure must carefully avoid intersecting with other structures and must avoid damaging other structures during channel formation processes.

For purposes herein, an “insulator” is a relative term that means a material or structure that allows substantially less (<95%) electrical current to flow than does a “conductor.” The dielectrics (insulators) mentioned herein can, for example, be grown from either a dry oxygen ambient or steam and then patterned. Alternatively, the dielectrics herein may be formed (grown or deposited) from any of the many candidate low dielectric constant materials (low-K (where K corresponds to the dielectric constant of silicon dioxide) materials such as fluorine or carbon-doped silicon dioxide, porous silicon dioxide, porous carbon-doped silicon dioxide, spin-on silicon or organic polymeric dielectrics, etc.) or high dielectric constant (high-K) materials, including but not limited to silicon nitride, silicon oxynitride, a gate dielectric stack of SiO₂ and Si₃N₄, hafnium oxide (HfO₂), hafnium zirconium oxide (HfZrO₂), zirconium dioxide (ZrO2), hafnium silicon oxynitride (HfSiON), hafnium aluminum oxide compounds (HfAlOₓ), other metal oxides like tantalum oxide, etc. The thickness of dielectrics herein may vary contingent upon the required device performance.

The conductors mentioned herein can be formed of any conductive material, such as polycrystalline silicon (polysilicon), amorphous silicon, a combination of amorphous silicon and polysilicon, and polysilicon-germanium, rendered conductive by the presence of a suitable dopant. Alternatively, the conductors herein may be one or more metals, such as tungsten, hafnium, tantalum, molybdenum, titanium, or nickel, or a metal silicide, any alloys of such metals, and may be deposited using physical vapor deposition, chemical vapor deposition, or any other technique known in the art.

There are various types of transistors, which have slight differences in how they are used in a circuit. For example, a bipolar transistor has terminals labeled base, collector, and emitter. A small current at the base terminal (that is, flowing between the base and the emitter) can control, or switch, a much larger current between the collector and emitter terminals. Another example is a field-effect transistor, which has terminals labeled gate, source, and drain. A voltage at the gate can control a current between source and drain. Within such transistors, a semiconductor (channel region) is positioned between the conductive source region and the similarly conductive drain (or conductive source/emitter regions), and when the semiconductor is in a conductive state, the semiconductor allows electrical current to flow between the source and drain, or collector and emitter. The gate is a conductive element that is electrically separated from the semiconductor by a “gate oxide” (which is an insulator); and current/voltage within the gate changes makes the channel region conductive, allowing electrical current to flow between the source and drain. Similarly, current flowing between the base and the emitter makes the semiconductor conductive, allowing current to flow between the collector and emitter.

Generally, transistor structures, in one example, can be formed by depositing or implanting impurities into a substrate to form at least one semiconductor channel region, bordered by shallow trench isolation regions below the top (upper) surface of the substrate. A “substrate” herein can be any material appropriate for the given purpose (whether now known or developed in the future) and can be, for example, silicon-based wafers (bulk materials), ceramic materials, organic materials, oxide materials, nitride materials, etc., whether doped or undoped. The “shallow trench isolation” (STI) structures are generally formed by patterning openings/trenches within the substrate and growing or filling the openings with a highly insulating material (this allows different active areas of the substrate to be electrically isolated from one another).

When patterning any material herein, the material to be patterned can be grown or deposited in any known manner and a patterning layer (such as an organic photoresist) can be formed over the material. The patterning layer (resist) can be exposed to some pattern of light radiation (e.g., patterned exposure, laser exposure, etc.) provided in a light exposure pattern, and then the resist is developed using a chemical agent. This process changes the physical characteristics of the portion of the resist that was exposed to the light. Then one portion of the resist can be rinsed off, leaving the other portion of the resist to protect the material to be patterned (which portion of the resist that is rinsed off depends upon whether the resist is a negative resist (illuminated portions remain) or positive resist (illuminated portions are rinsed off). A material removal process is then performed (e.g., wet etching, anisotropic etching (orientation dependent etching), plasma etching (reactive ion etching (RIE), etc.)) to remove the unprotected portions of the material below the resist to be patterned. The resist is subsequently removed to leave the underlying material patterned according to the light exposure pattern (or a negative image thereof).

While only one or a limited number of transistors are illustrated in the drawings, those ordinarily skilled in the art would understand that many different types transistor could be simultaneously formed with the embodiment herein and the drawings are intended to show simultaneous formation of multiple different types of transistors; however, the drawings have been simplified to only show a limited number of transistors for clarity and to allow the reader to more easily recognize the different features illustrated. This is not intended to limit this disclosure because, as would be understood by those ordinarily skilled in the art, this disclosure is applicable to structures that include many of each type of transistor shown in the drawings.

The terminology used herein is for the purpose of describing particular embodiments only and is not intended to be limiting of the foregoing. As used herein, the singular forms “a,” “an,” and “the” are intended to include the plural forms as well, unless the context clearly indicates otherwise. Furthermore, as used herein, terms such as “right”, “left”, “vertical”, “horizontal”, “top”, “bottom”, “upper”, “lower”, “under”, “below”, “underlying”, “over”, “overlying”, “parallel”, “perpendicular”, etc., are intended to describe relative locations as they are oriented and illustrated in the drawings (unless otherwise indicated) and terms such as “touching”, “in direct contact”, “abutting”, “directly adjacent to”, “immediately adjacent to”, etc., are intended to indicate that at least one element physically contacts another element (without other elements separating the described elements).

Each respective figure, in addition to illustrating methods of and functionality of the present embodiments at various stages, also illustrates the logic of the method as implemented, in whole or in part, by one or more devices and structures. Such devices and structures are configured to (i.e., include one or more components, such as resistors, capacitors, transistors and the like that are connected to enable the performing of a process) implement the method described above. In other words, one or more computer hardware devices can be created that are configured to implement the method and processes described herein with reference to the FIGs. and their corresponding descriptions.

Embodiments herein may be used in a variety of electronic applications, including but not limited to advanced sensors, memory/data storage, semiconductors, microprocessors and other applications. A resulting device and structure, such as an integrated circuit (IC) chip can be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

The corresponding structures, materials, acts, and equivalents of all means or step plus function elements in the claims below are intended to include any structure, material, or act for performing the function in combination with other claimed elements as specifically claimed. The description of the present embodiments has been presented for purposes of illustration and description but is not intended to be exhaustive or limited to the embodiments in the form disclosed. Many modifications and variations will be apparent to those of ordinary skill in the art without departing from the scope and spirit of the embodiments herein. The embodiments were chosen and described in order to best explain the principles of such, and the practical application, and to enable others of ordinary skill in the art to understand the various embodiments with various modifications as are suited to the particular use contemplated.

While the foregoing has been described in detail in connection with only a limited number of embodiments, it should be readily understood that the embodiments herein are not limited to such disclosure. Rather, the elements herein can be modified to incorporate any number of variations, alterations, substitutions or equivalent arrangements not heretofore described, but which are commensurate with the spirit and scope herein. Additionally, while various embodiments have been described, it is to be understood that aspects herein may be included by only some of the described embodiments. Accordingly, the claims below are not to be seen as limited by the foregoing description. A reference to an element in the singular is not intended to mean “one and only one” unless specifically stated, but rather “one or more.” All structural and functional equivalents to the elements of the various embodiments described throughout this disclosure that are known or later, come to be known, to those of ordinary skill in the art are expressly incorporated herein by reference and intended to be encompassed by this disclosure. It is therefore to be understood that changes may be made in the particular embodiments disclosed which are within the scope of the foregoing as outlined by the appended claims.

What is claimed is:

1. A structure comprising:
a first layer, wherein the first layer includes a recess;
an intermediate layer contacting the first layer; and
a contact-free biosensor aligned above the recess,
wherein a portion of the intermediate layer is positioned along the recess, and
wherein the portion of the intermediate layer that is positioned along the recess separates the contact-free biosensor from the recess.

2. The structure according to claim 1, wherein the contact-free biosensor comprises one or more of:
a transistor device;
a transmission line; and
an antenna.

3. The structure according to claim 1, wherein the recess comprises a plurality of recesses positioned across the intermediate layer from the contact-free biosensor.

4. The structure according to claim 1, further comprising a plurality of dividers within the recess.

5. The structure according to claim 1, wherein the contact-free biosensor is positioned relative to the recess in a location to detect characteristics of a fluid under test within the recess using a time-varying electromagnetic field at least one of generated and received by the contact-free biosensor.

6. The structure according to claim 1, further comprising a fluid receptacle connected to the recess, wherein the recess is shaped and sized to transport fluid under test supplied to the fluid receptacle past the contact-free biosensor.

7. The structure according to claim 1, wherein the first layer comprises a bulk wafer substrate, and wherein the intermediate layer comprises at least one of:
a buried oxide layer;
a silicon layer;
a silicon oxide layer; and
a silicon germanium layer.

8. A structure comprising:
a first layer, wherein the first layer includes a recess;
an intermediate layer contacting the first layer; and
a plurality of contact-free biosensors aligned above the recess,
wherein a portion of the intermediate layer is positioned along the recess, and
wherein the portion of the intermediate layer that is positioned along the recess separates the contact-free biosensors from the recess.

9. The structure according to claim 8, wherein the contact-free biosensors comprise one or more of:
transistor devices;
transmission lines; and
antennas.

10. The structure according to claim 8, wherein the recess comprises a plurality of recesses positioned across the intermediate layer from the contact-free biosensors.

11. The structure according to claim 8, further comprising a plurality of dividers within the recess.

12. The structure according to claim 8, wherein the contact-free biosensors are positioned relative to the recess in locations to detect characteristics of a fluid under test within the recess using a time-varying electromagnetic field at least one of generated and received by the contact-free biosensors.

13. The structure according to claim 8, further comprising a fluid receptacle connected to the recess, wherein the recess is shaped and sized to transport fluid under test supplied to the fluid receptacle past the contact-free biosensors.

14. The structure according to claim 8, wherein the first layer comprises a bulk wafer substrate, and wherein the intermediate layer comprises at least one of:
a buried oxide layer;
a silicon layer;
a silicon oxide layer; and
a silicon germanium layer.

15. A method comprising:
forming a first layer to include a recess;
forming an intermediate layer to contact the first layer; and
forming a contact-free biosensor aligned above the recess,
wherein a portion of the intermediate layer is formed to be positioned along the recess, and
wherein the portion of the intermediate layer that is positioned along the recess is formed to separate the contact-free biosensor from the recess.

16. The method according to claim 15, wherein the contact-free biosensor is formed as one or more of:
a transistor device;
a transmission line; and
an antenna.

17. The method according to claim 15, wherein formation of the recess comprises forming a plurality of recesses positioned across the intermediate layer from the contact-free biosensor.

18. The method according to claim 15, further comprising forming a plurality of dividers within the recess.

19. The method according to claim 15, wherein the contact-free biosensor is formed to be positioned relative to the recess in a location to detect characteristics of a fluid under test within the recess using a time-varying electromagnetic field at least one of generated and received by the contact-free biosensor.

20. The method according to claim 15, further comprising forming a fluid receptacle connected to the recess, wherein the recess is formed to be shaped and sized to transport fluid under test supplied to the fluid receptacle past the contact-free biosensor.
